(19) United States
(12) Patent Application Publication
(10) Pub. No.: US 2017/0143930 A1
(43) Pub. Date: May 25, 2017

(54) METHOD AND APPARATUS FOR MAINTAINING PATIENT BODY TEMPERATURE DURING SURGERY

(71) Applicants: Cray V. Noah, Spring, TX (US); Rady Villaflor, Spring, TX (US)

(72) Inventors: Cray V. Noah, Spring, TX (US); Rady Villaflor, Spring, TX (US)

(21) Appl. No.: 15/354,101

(22) Filed: Nov. 17, 2016

Related U.S. Application Data
(60) Provisional application No. 62/257,259, filed on Nov. 19, 2015.

(51) Int. Cl. A61M 16/10; A61M 16/16

(52) U.S. Cl. CPC A61M 16/1095; A61M 16/16; A61M 2205/3368; A61M 2205/52; A61M 2205/366; A61M 2209/082

(57) ABSTRACT

A method for maintaining body temperature includes heating a volume disposed externally to a conduit forming an inspiratory limb of a respirator. Gas is moved through the conduit to a patient using the respirator for inhalation of the moved gas by the patient.

### CROSS REFERENCE TO RELATED APPLICATIONS

Priority is claimed from U.S. Provisional Application No. 62/257,259 filed on Nov. 19, 2015 and incorporated herein by reference in its entirety.

### Statement Regarding Federally Sponsored Research of Development

Not Applicable.

### NAMES TO THE PARTIES TO A JOINT RESEARCH AGREEMENT

Not Applicable.

### BACKGROUND

This disclosure relates generally to the field of apparatus used in surgical medical procedures. More specifically, the disclosure relates to methods and apparatus for maintaining patient body core temperature, for example, during surgical procedures.

It is known that human body core temperature drops significantly during surgery for three main reasons: (i) the physical environment of the operating room is cold, on average these rooms are maintained at ambient temperatures of 20-22° C.; (ii) general anesthetics inhibit the human body's natural temperature maintenance mechanisms; and (iii) exposed, lacerated portions of the human body lose significant amounts of heat to the ambient surroundings. Additionally, certain medical authorities have explained that within the first hour of administration of anesthesia, patients experience vasodilation—and the consequent movement of blood to the peripheries of the body. Thus, blood is transported away from the body core, the most crucial area for sustaining heat in the body because it provides warmth to the vital organs. Lower core body temperature may lead to hypothermia more quickly than thermodergulating any other portion of the body. See, Sessler, D. I. (2008), Temperature Monitoring and Perioperative Thermoregulation, Anesthesiology, 109(2), 318-338, doi:10.1097/ALN.0b013e31817f6d76. Thus, along with the above mentioned factors, blood redistribution via vasodilation appears also to exacerbate heat loss, and in the essential region of the body core specifically. Some patients are at an even greater risk of developing intraoperative hypothermia due to factors such as age, low body mass, inefficient blood circulation, previous adverse medical conditions, etc.

Because even mild hypothermia is known to correlate with delayed wound healing and post-anesthetic recovery, prolonged hospitalization, and increased patient discomfort (see, Sessler, 2008 and Frank S. M., Fleisher L. A., Breslow M. J., Higgins M. S., Olson K. F., Kelly S., Beattie C. (1997), Perioperative maintenance of normothermia reduces the incidence of morbid cardiac events: A randomized clinical trial. JAMA, 277: 1127-34), there is a need for an effective intrasurgical warming mechanism that targets the body core. The issue of insubstantial intrasurgical warming has recently gained priority, marked by instatement of The Surgical Care Improvement Project (SCIP), which mandates penalties for anesthesiologists who do not abide by patient normothermia standards, specifically, achieving a postsurgical patient core body temperature of at least 36° C.

A widely used surgical patient warming device sold under the trademark BAIR

HUGGER® is believed to be an inadequate device for maintaining patient normothermia. Due to the blanket/cover form of the BAIR HUGGER patient warming device, the device may be obstructive and not translatable to many surgical procedures. There is also believed to be risk of contamination due to forced airflow and proximity to the sterile zone (see, Legg et al. Forced-air patient warming blankets disrupt unidirectional airflow, Bone and Joint Journal, 2013; 95-B:407-410). Further, there have been instances of patient tissue burning. See, Chung, K., Lee, S., Oh, S. C., Choi, J., & Cho, H. S. (2012), Thermal burn injury associated with a forced-air warming device, Korean Journal of Anesthesiology, 62(4), 391-392. doi:10.4097/kjae.2012.62.4.391 and Truell, K. D., Bakerman, P. R., Teodori, M. F., & Maze, A. (2000), Third-degree burns due to intraoperative use of a BAIR HUGGER warming device, Annals of Thoracic Surgery, 69(6), 19331934. doi:10.1016/S0003-4975(00)01322-9). BAIR HUGGER is a registered trademark of 3M Corp., 10393 West 70th Street, Suite 100 Eden Prairie Minn. 55344.

Anesthesiologists require a simple, effective, and non-obstructive device that can weather the hectic environment of the operating room (OR) and maintain patient core body temperatures in any surgical procedure; because the aforementioned problems are inherent to the concept of a blanket or covering, an alternative method to intraoperative warming was sought.

### BRIEF DESCRIPTION OF THE DRAWINGS

FIG. 1A shows an example assembled view of an example embodiment of an apparatus according to the present disclosure.

FIG. 1 shows the apparatus of FIG. 1A with the conduit removed.

FIG. 2 shows an exploded view of the example embodiment shown in FIG. 1.

FIG. 3 shows an example embodiment of the apparatus attached to the inspiratory limb (conduit) of an anesthesia gas delivery system.

### DETAILED DESCRIPTION

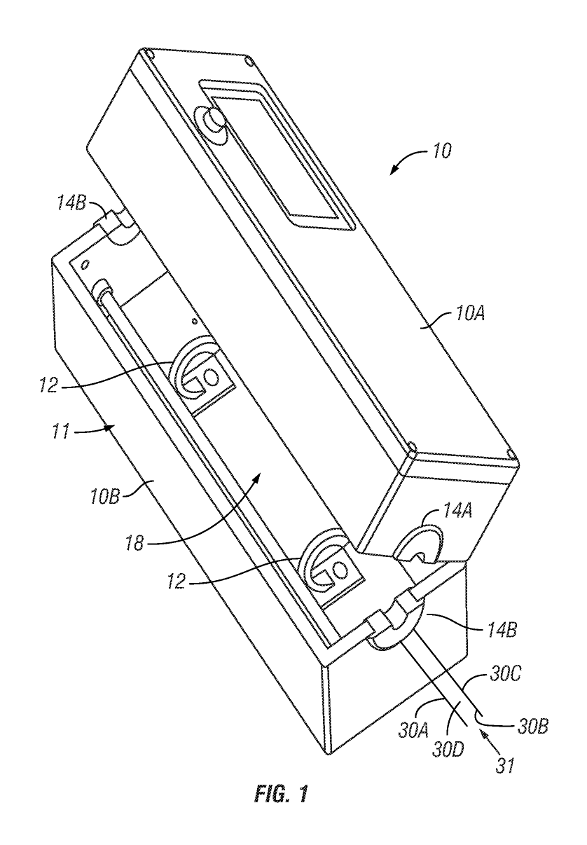

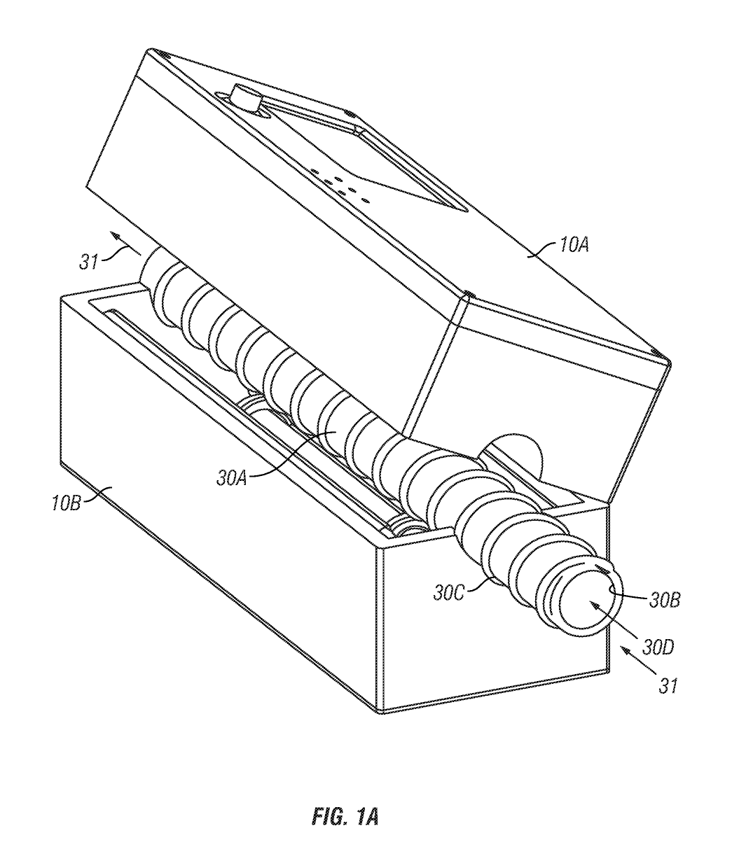

FIG. 1A shows an assembled, open view of an example embodiment of a patient body temperature warming apparatus according to the present disclosure. While the present disclosure is explained in terms of an apparatus and method usable with surgery, it should be understood that a method and apparatus according to the present disclosure are not limited to such uses.

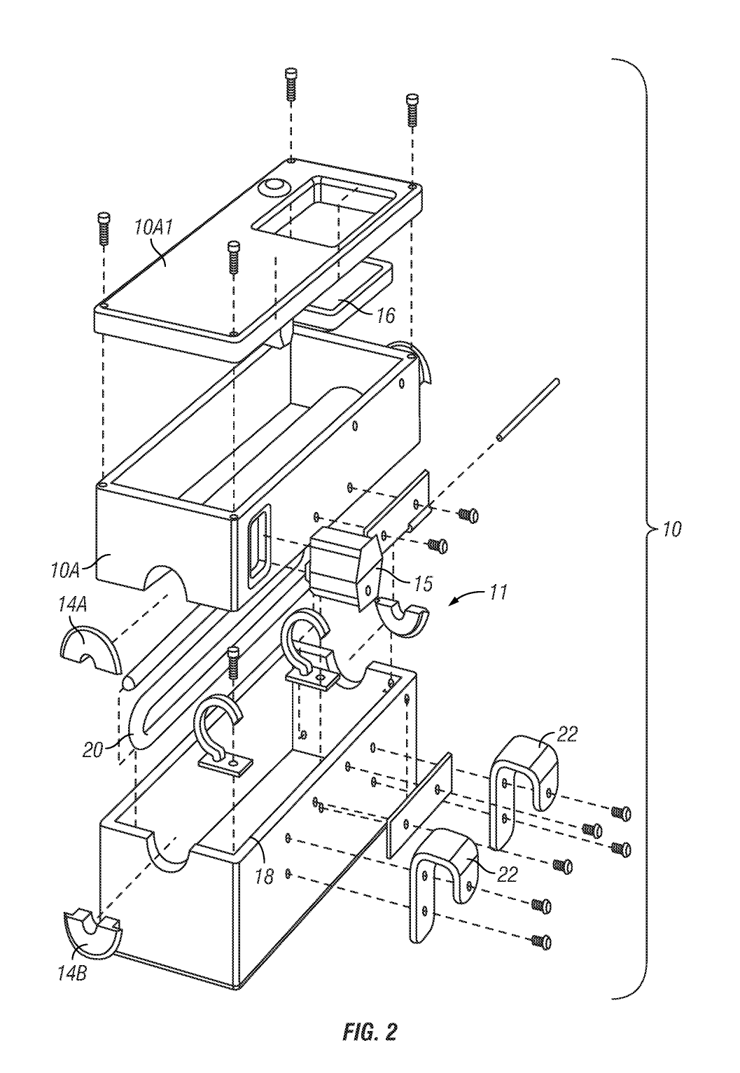

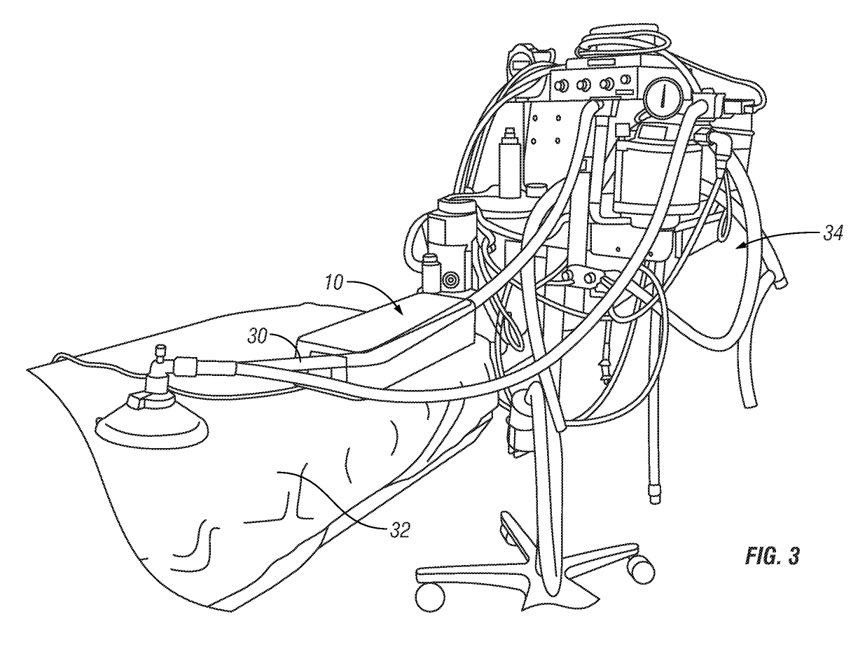

Gas, shown schematically at 31 to be inhaled by a patient (not shown) is moved through a conduit 30A of an inspiratory limb (30 in FIG. 3) of a respirator (see 34 in FIG. 3) for inhalation by the patient. The conduit 30A has a predetermined wall thickness (defined as a difference between an external and internal diameter of the conduit 30A) with an internal surface 30B defining a passageway 30D for the gas 31, and an external surface 30C opposite the internal surface 30B. At least a portion of the external surface 30C of the conduit 30A is heated with a heat source (e.g., heating element 20 in FIG. 2) located outside the passageway 30D. The external surface 30C continues to be heated until heat transfers from the external surface 30C through the wall thickness of the conduit 30A to heat the gas 31.

FIG. 1 shows the apparatus 10 with the conduit 30A excluded to show some internal features. In the present example embodiment, the apparatus 10 may be disposed in a container 11, for example a hingedly closed, rectangularly shaped box, although the exact shape of the container 11 is not a limit on the scope of possible embodiments according to the present disclosure. The container or box 11 may include an upper cover 10A hingedly connected to a lower housing 10B in which a predetermined internal volume 18 may be defined by the lower housing 10B. Hose clamps 12 may be affixed to an interior surface of the lower housing 10B such that an inspiratory limb or conduit (see FIG. 3) of an anesthetic gas supply system (a respirator) may be removably affixed. Ends of the container 11 may be sealed by corresponding upper end seals 14A insertable into correspondingly shaped openings in the upper cover 10A and lower end seals 14B insertable into corresponding openings in the lower housing 10B such that when the inspiratory limb (e.g., conduit 30A in FIG. 1A or shown at 30 in FIG. 3) is disposed inside the lower housing 10B and the upper cover 10A is closed, the end seals 14A and 14B sealingly engage the inspiratory limb (FIG. 3) where it passes through the container 11.

An exploded view of the example embodiment of the apparatus is shown in FIG.

2, wherein may be further observed a heating element 20 disposed in the volume 18, which may be an electric resistance heating element. The power rating of the heating element 20 may be selected based on the expected flow of anesthetic and respiratory gases passed through the conduit (30A in FIG. 1) and the temperature to which such gases are to be heated and maintained during operation of the apparatus. A control panel 16 may be accessible through an opening 10A1 in the upper cover 10A. The control panel 16 may include a controller (not shown separately) such as a programmable logic controller that is in signal communication with a temperature sensor (not shown) and controls power output to the heating element 20, for example, using a potential integral derivative (PID) control loop programmed into the controller or provided as a separate element in signal communication with the controller. The user may set an operating temperature of fluid, e.g., water, to be disposed in the lower housing 10B such that when clamped in place by the hose clamps (12 in FIG. 1), the inspiratory limb and the heating element are submerged in water. A sealed electrical connector 15 may provide electrical power through the wall of the container 11 for operating the control panel 16 and the heating element 20.

In other embodiments, the heating element 20 and the conduit (30A in FIG. 1) may be disposed in air inside the volume 18 such that heating the gas (31 in FIG. 1) inside the conduit (30A in FIG. 1) is performed by radiative heat transfer. In other embodiments, the heating element 20 may be, for example, electrical resistance wire embedded in a flame resistant cloth member to form a heating blanket which may be wrapped on the external surface (30C in FIG. 1) of at least part of the conduit (30A in FIG. 1) to heat the gas (31 in FIG. 1) flowing therein by conductive heat transfer.

Clamp hooks 22 or similar devices may be affixed to the side of the container 11 to enable suspension thereof from a convenient portion of a patent operating bed (FIG. 3), whereby the apparatus 10 may be placed out of the way of the personnel conducting a surgical procedure.

To set up and use the apparatus, the anesthesiologist or a person acting under his direction in the case of a surgical procedure closes the upper cover 10A and lower housing 10B of the container 11 around a portion of the inspiratory limb (FIG. 3) such that the inspiratory limb (FIG. 3) is disposed within the container 11 and exits through the seals 14A, 14B on opposed ends of the container 11. The clamps 12 on the inside, bottom surface of the lower housing 10B effectively keep this portion of the inspiratory limb submerged in a fluid, e.g., water, which may fill the volume 18. Next, the clamp hooks 22 located on the apparatus may be used to secure it to the railing of a patient operating bed (FIG. 3) or in any other convenient location for compactness. Lastly, the anesthesiologist or person acting under his direction operates the apparatus 10 to heat the fluid inside the volume 18 to an uppermost temperature of about 45 degrees C. The anesthesiologist may then allow the apparatus 10 to operate automatically. As explained above, the controller 16 may use a proportional integral derivative (PID) control loop to regulate the temperature of the fluid and the heating element 20 within the container 11. Other control methods may be used, including a simple thermostat that measures the temperature of the fluid in the volume 18 and switches the heating element 20 on and off to maintain the fluid in the volume 18 at a setpoint temperature. The selected water temperature may be initially selected by the anesthesiologist. The heated fluid (e.g., water) in turn warms the gases flowing through the submerged portion of the inspiratory limb (FIG. 3) to that set temperature or slightly lower (a relationship between setpoint temperature of the fluid and the final temperature of the flowing gases can be quantified as shown in the following sections). Because the conduit (30A in FIG. 1) is completely submerged in fluid inside the apparatus 10, the surface area in contact with the fluid is constant, and thus the moving gases are efficiently heated without using large amounts of heat or high temperatures to transfer heat from the heating element 20 to the inspiratory limb. The foregoing warmed gases effectively warm the patient upon inspiration.

Engineering analysis was performed to identify the feasibility of heating the gases flowing through the anesthesia circuit (inspiratory limb) from 22° C. ambient room temperature to 37° C. using a heated water bath. Starting with the convection equation, the amount of power needed to obtain the desired 15° C. temperature change was determined. Using that value, a Reynolds number was determined for the flowing gases which indicated that the flow of gases was substantially laminar for ordinary inspiratory tube sizes and gas flow rates. This enabled estimating Nusselts number, a value obtained from Incropera, F., & DeWitt, D. (2002). Fundamentals of heat and mass transfer (5th ed.). New York: J. Wiley and Sons, Inc. Lastly, the above entioned values were used to compute the heat transfer coefficient of moving gases, finally enabling determining the length of tubing needed to be submerged in fluid to obtain the required flowing gas heating. It was determined that at 46° C. water temperature, the required tube length was 0.9 meters, and thus determined a water bath heating mechanism was viable and feasible for the present example embodiment.

Volumetric flow rate experiments to confirm the functionality of the prototype were controlled using a 12 volt laptop computer fan to move gases through a tube and a motor speed controller to control the gas flow rate. The velocity of air was measured with an air velocity meter to confirm it was flowing at the same rate as would be the case in an anesthesia circuit, i.e., about 0.4 m/s. The water in a thermally insulated box was heated with a 1000 watt heating element connected to a PID controller, which modulated the amount of heat from the heating element (20 in FIG. 2) to maintain the set temperature. From these experiments, it was determined that the optimal length of tubing/water bath temperature combination for reaching 37° C. gas temperature was 38 cm, or about 15 inches in a water bath of temperature 46° C., a process that took just four minutes. Though the general concept of warming gases in the anesthesia circuit has been utilized before by warming humidifiers that act directly on the gases, heating the inspiratory limb externally allows for easily retrofitting existing inspiratory limbs without creating a risk of contaminating the gas inhaled by the patient. By keeping the functional components of the apparatus on the outside of the anesthesia circuit, sterility is not an issue, and one may take advantage of readily available disposable anesthesia limbs. Proof of concept experimentation shows the device does indeed heat and sustain the flowing air temperature at 37° C., body temperature, and thus will effectively warm the core temperature of patients during surgery. Because the minimum air temperature for producing a burn in the lungs is 45° C. sustained for 5 hours (see, Rao, D. (n.d.), Thermal Injuries|Forensic Pathology Online, Retrieved Jul. 22, 2015.), the apparatus is believed to be safe, as the warm water bath never exceeds 46° C., and due to natural heat loss, the air in the inspiratory limb may be expected not to exceed 44° C. The apparatus is also non-obstructive, as it can be hooked onto the railing of an operating bed, is translatable to all surgical procedures, features a simple interface for the user, and is operable in a “set and forget” fashion. Also, because of the phenomena of vasodilation previously described, warming the core body temperature is a possible substantial advantage in maintaining patient core body temperature during surgery.

FIG. 3 shows a prototype of the apparatus 10 clamped around an inspiratory limb 30 of an anesthesia respirator 34. The small relative size of the apparatus 10 relative to the size of the operating table 32 (i.e., the patient bed) is apparent, whereby the apparatus 10 may be used with minimal disruption to medical procedures being performed on a patient.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method for maintaining body temperature, comprising:
heating a volume disposed externally to a conduit forming an inspiratory limb of a respirator; and
moving gas through the conduit to a patient using the respirator for inhalation of the moved gas by the patient.

2. The method of claim 1 wherein the conduit is heated by conductive heat transfer.

3. The method of claim 2 wherein the conductive heat transfer is performed using fluid in contact with an exterior of the conduit.

4. The method of claim 3 wherein the fluid comprises water.

5. The method of claim 1 wherein the volume is heated by an electric heating element.

6. The method of claim 5 wherein a temperature of the electric heating element is controlled by a proportional integral derivative controller.

7. The method of claim 1 wherein a temperature in the volume is at least equal to normal body temperature.

8. The method of claim 1 wherein a temperature in the volume is selected such that gases flowing through the conduit are heated to at most 45 degrees C.

9. An apparatus for maintaining body temperature, comprising:
a container for sealingly engaging an exterior of a conduit forming an inspiratory limb of a respirator;
a heating element and a fluid disposed in a volume defined by the enclosure;
means for retaining the conduit submerged in the fluid; and
means for controlling temperature of the fluid in signal communication with the heating element.

10. The apparatus of claim 9 wherein the heating element comprises an electric heating element.

11. The apparatus of claim 9 wherein the container comprises an upper cover and a lower housing hingedly connected to each other and seals engageable with the conduit when the upper cover and the lower housing are closed.

12. The apparatus of claim 9 wherein the means for controlling temperature of the fluid comprises a proportional integral derivative controller.

13. The apparatus of claim 9 wherein the means for controlling temperature maintains the fluid at a preselected temperature.

14. The apparatus of claim 13 wherein the preselected temperature is at most 46 degrees C.

15. The apparatus of claim 9 further comprising retaining hooks disposed on an exterior of the container for suspending the container on a bed rail.

16. A method for heating gas to be inhaled by a patient, comprising:
moving gas through a conduit of an inspiratory limb of a respirator for inhalation by a patient, the conduit having a wall thickness with an internal surface defining a passageway for the gas, and an external surface opposite the internal surface;
heating at least a portion of the external surface of the conduit with a heat source located outside the passageway;
continuing to heat the external surface until heat transfers from the external surface through the wall thickness to heat the gas.

17. The method of claim 16 wherein the heat source comprises a heating blanket in contact with the at least a portion of the external surface.

18. The method of claim 16 wherein the heat source comprises a fluid in contact with at least a portion of the external surface.

19. The method of claim 16 wherein the heat source is a source of radiative heat transfer directed at the external surface.

20. The method of claim 16 wherein a temperature of the heat source is controlled by a proportional integral derivative controller.

21. The method of claim 16 wherein a temperature of the gas is maintained at least equal to normal body temperature.

22. The method of claim 1 wherein a temperature of the heat source is selected such that gases flowing through the conduit are heated to at most 45 degrees C.
